(19) United States
(12) Patent Application Publication
(10) Pub. No.: US 2024/0074744 A1
(43) Pub. Date: Mar. 7, 2024

(54) RETRACTION DEVICE FOR MINIMALLY INVASIVE SURGERY

(71) Applicant: Flexbar Machine Corp., Islandia, NY (US)

(72) Inventors: Danny Sherwinter, Brooklyn, NY (US); Benjamin M. Nawrath, Holbrook, NY (US); Steven Culver, Stony Brook, NY (US)

(21) Appl. No.: 18/459,634

(22) Filed: Sep. 1, 2023

Related U.S. Application Data
(60) Provisional application No. 63/374,563, filed on Sep. 4, 2022.

(51) Int. Cl. A61B 17/02

(52) U.S. Cl. CPC A61B 17/0293; A61B 2017/00858

(57) ABSTRACT

A retraction device that can be used inside a patient during a surgical procedure is provided. The retraction device includes pivotally connected links and connection ends such that the ring can be flattened and introduced into a patient laparoscopically. Once introduced to a laparoscopic surgery site, the retraction device can be formed and used while within the patient. Once use is complete, the device that can be in the form of a ring can be substantially flattened and removed from the patient using the laparoscopic tool it was introduced with.

### BACKGROUND

### Technical Field

The present invention relates to retraction devices and rings used during surgery. More particularly, it relates to retraction devices that can be used inside a patient during a surgical procedure.

### Description of Related Art

In standard surgical practice there is a need for retraction of tissue so as to obtain optimum positioning and/or visualization of the tissue to be worked on. In open surgery this can be accomplished readily due to the extensive availability of space and the almost infinite number of instruments and retractors that can be positioned in the surgical field (over the patient). In contrast, during laparoscopic/minimally invasive surgery, access to the abdominal cavity is, by definition, constrained to a limited number of small incisions. Although leading to better outcomes for the patients, this limits the surgeons' ability to obtain adequate retraction of tissue restraining visualization and or access to the tissue of interest. Surgeons often struggle to use the limited instrumentation available to them within the patient's surgical cavity which limits their ability to retain optimal visualization, access, and angle of attack.

Thus, there is clearly a need for a device that can be placed into the abdominal cavity through the small incisions inherent to minimally invasive surgery yet has the ability to provide excellent retraction. A device such as described does not currently exist.

### SUMMARY OF THE INVENTION

In accordance with one implementation, the retraction device for minimally invasive surgery includes a plurality of links pivotally connected to each other. The plurality of links form a chain of links having opposing ends. A connection mechanism is positioned at each of the opposing ends and is configured to connect the opposing ends and cause the plurality of links to form the retraction device. The plurality of links are introduced into a patient in a substantially flat configuration prior to engagement of the connection mechanism.

In accordance with another implementation, the plurality of links include a slit for receiving and securing at least one of a suture or a retraction hook.

In alternative implementations, the slit has a variable size and at least two links of the plurality of links have slits of a different size with respect to each other.

According to yet another aspect, at least one of the plurality of links includes a textured surface to assist in handling and manipulation of the ring during use.

In accordance with other implementations, the connection mechanism can take various forms. For example, magnets disposed in or on the opposing ends of the plurality of links. Another example includes a hook disposed on each of the opposing ends of the plurality of links, where a suture is wrapped around the hooks on each opposing end to hold and secure the opposing ends together. In another implementation the connection mechanism includes a socket at one of the opposing ends and a press fit end having a locking rib disposed around the same such that when the locking rib engages the socket, the ends are secured together.

In a further embodiment, the retraction device includes a channel formed in the plurality of links, and a wire positioned in the channel. The wire is fixed at one of the opposing ends and extends out of the channel at the other of the opposing ends. The wire enables the formation of the retraction device by pulling on the extended end which brings the opposing ends together.

In yet a further embodiment, the retraction device for use inside a patient during minimally invasive surgery includes at least two links connected to each other and having opposing ends. Each of the at least two links has at least one slit configured to receive and secure a suture or a retraction hook. The at least two links can be laparoscopically introduced into a patient in a substantially flat configuration prior to engagement of a connection mechanism.

Other aspects and features of the present principles will become apparent from the following detailed description considered in conjunction with the accompanying drawings. It is to be understood, however, that the drawings are designed solely for purposes of illustration and not as a definition of the limits of the present principles, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings wherein like reference numerals denote similar components throughout the views:

FIG. 1A is a representation of the retraction device in a flattened state according to one embodiment;

FIG. 1B is a plan view of the formation of the retraction device of FIG. 1 according to an embodiment;

FIG. 1C shows a perspective view of the retraction device according to an embodiment;

FIG. 1D shows a perspective view of the retraction device according to another embodiment;

FIG. 1E shows a perspective view of the retraction device according to another embodiment;

FIG. 2 is a plan view of the retraction device shown assembled according to yet another embodiment;

FIGS. 3A-3M show different embodiments of the connection mechanism between the ends of the retraction device;

FIG. 4 is a plan view showing how the retraction device can be inserted into a patient using a trocar, according to one embodiment;

FIG. 5A is a perspective view of the retraction device being introduced into a patient via a trocar medical device according to an embodiment;

FIG. 5B is a perspective view showing the retraction device as it would be formed within the patient after insertion using a device like that shown in FIG. 5A according to an embodiment;

FIGS. 6A-6C are plan views showing examples of the retraction device within a patient and the use of the same, according to an embodiment; and

FIGS. 7A-7C show an alternative implementation of a collapsible retraction device according to another emodiment.

### DETAILED DESCRIPTION

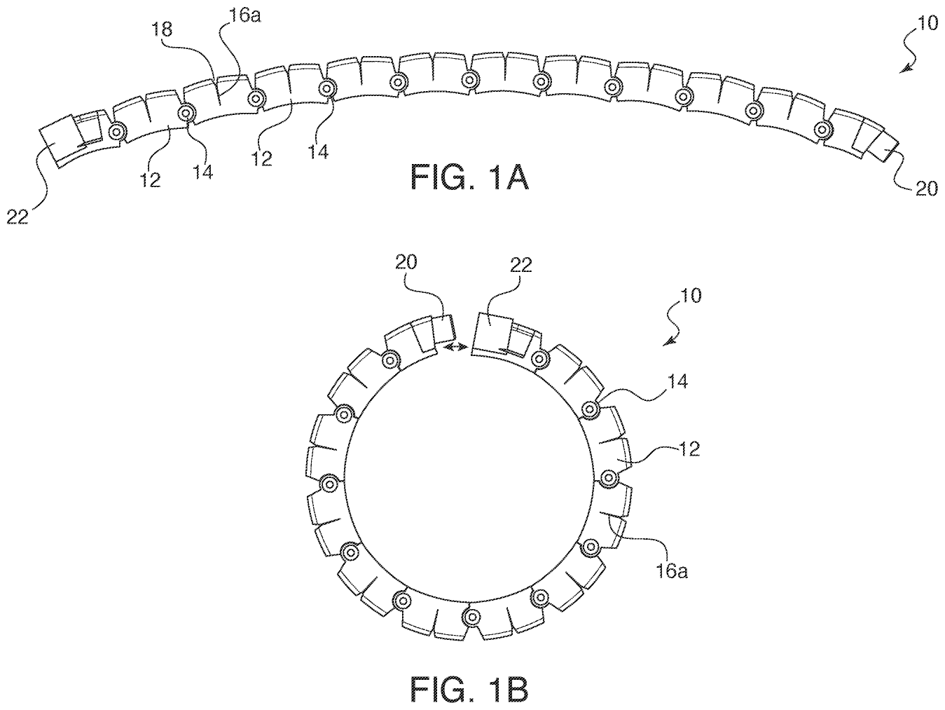

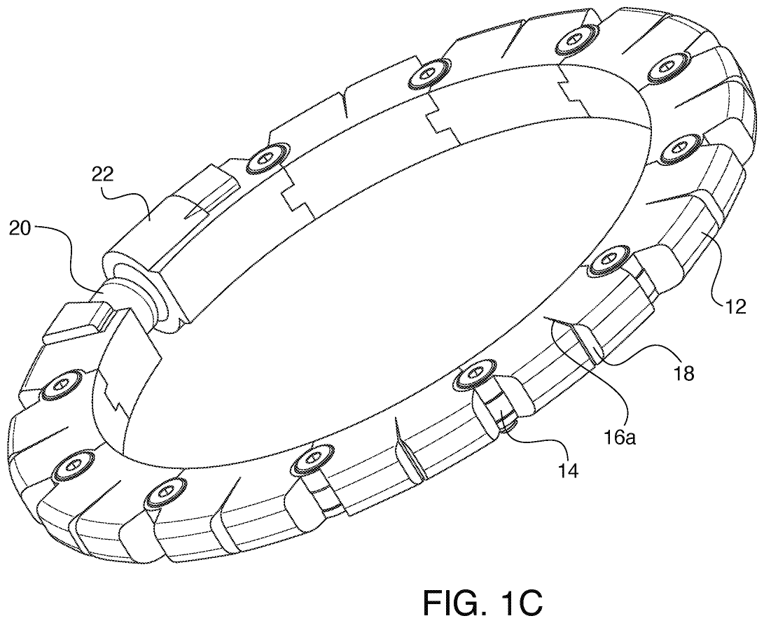

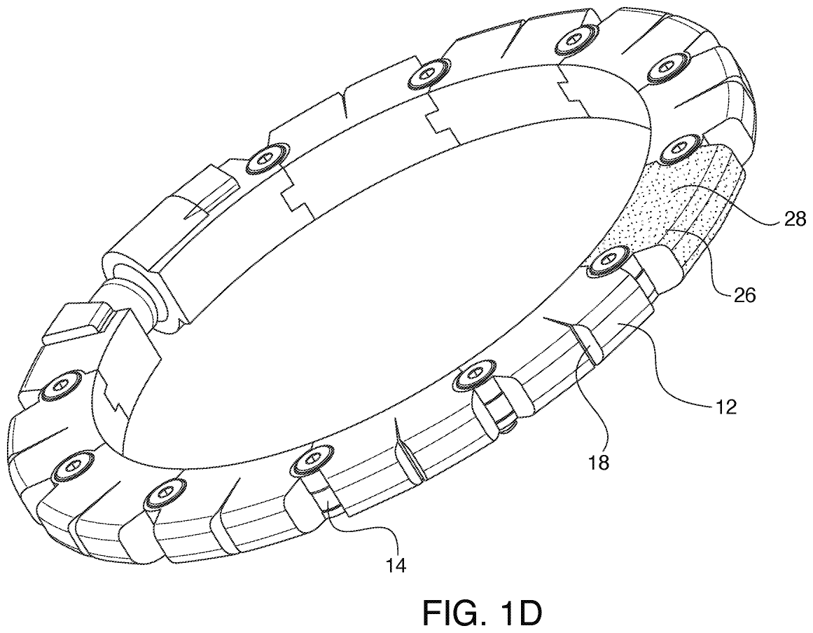

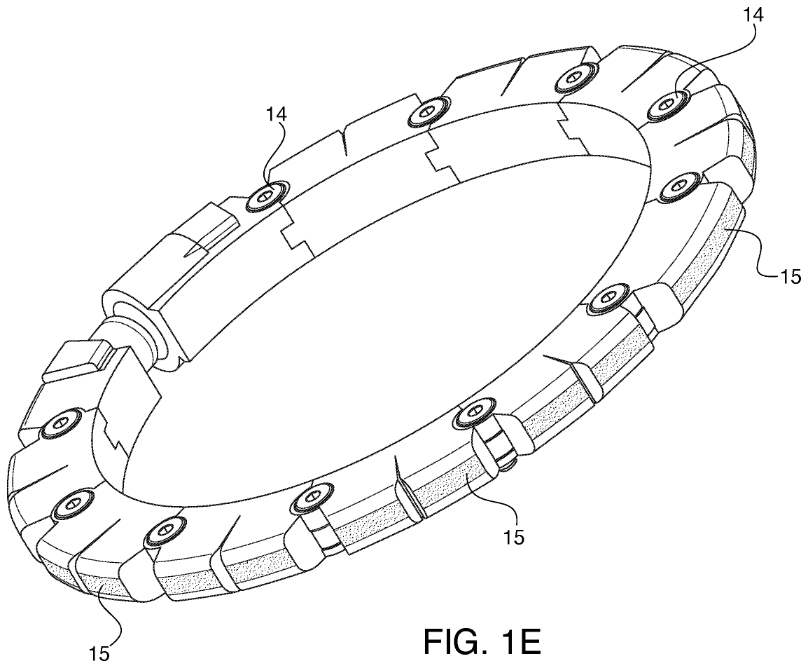

FIGS. 1A-1C show one embodiment of the internal retraction or retention device 10 according to an embodiment. The retraction device 10 is made up of a rigid material (e.g., hard plastics, metals, etc.) in the form of a plurality of links 12 that are pivotally connected 14 to each other to form a chain of links such that when the ends 20 and 22 are brought together a “ring” is substantially formed. The “ring” is flexible in nature yet the component parts or links are rigid and can be formed from metals such as, for example, stainless steel, aluminum. As shown in the present exemplary embodiment, each of the links 12 have a shape and corresponding geometry such that the desired number of links 12 can be introduced into a patient in a substantially flattened or straightened form, and then can form a ring as shown when the ends are brought together. In accordance with other implementations, the links 12 can have a shape and corresponding geometry that when linked or connected together form an oval, ellipse, a square, a triangle or any other desired shape for a retraction device/ring application. In the following description, the retraction device 10 is shown primarily as a ring, yet as mentioned, other shapes and forms can be made of the retraction device in accordance with the present principles.

As will be appreciated by those of skill in the art of laparoscopic surgery, the diameter of the retraction device 10, once assembled inside a patient can be, for example, in a range of 1 in −5 inches. It will be appreciated that different sizes can be implemented depending on the desired application and/or surgical procedure. In accordance with a preferred implementation, the retraction device 10 and all components thereof are designed to be a one use, disposable item, thus eliminating the need or concern for sterilization after use.

In accordance with other implementations, the retraction device and all components thereof are designed to be re-used.

In accordance with further embodiments the number of links can be varied depending on their respective elasticity and/or the desired shape of the device and the intended application.

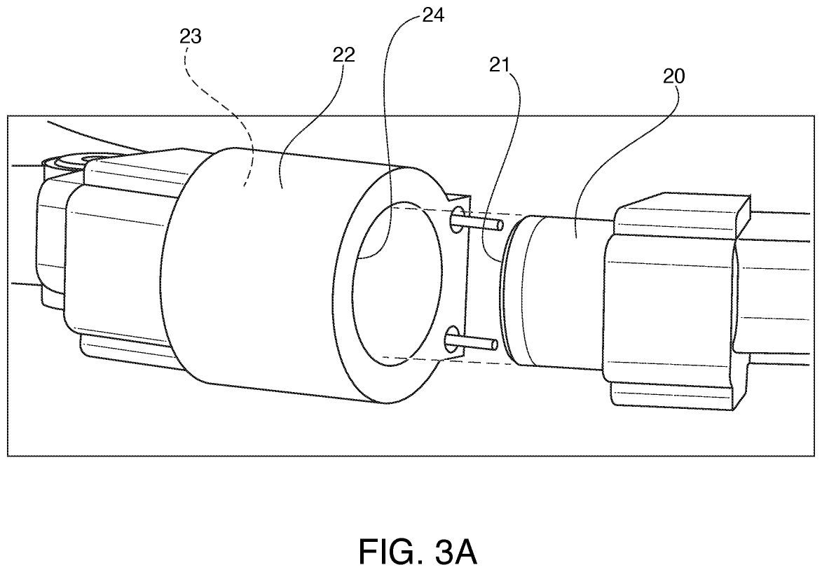

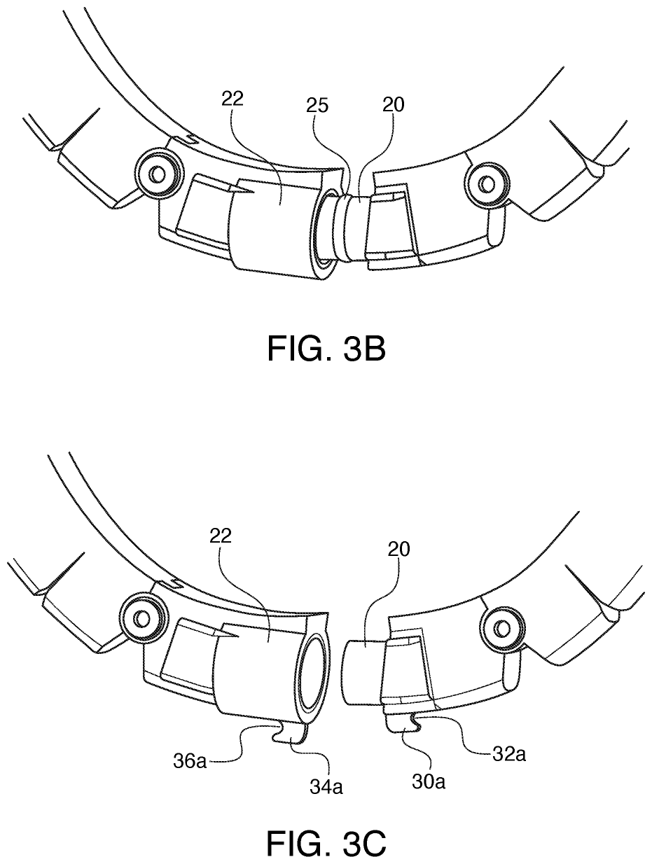

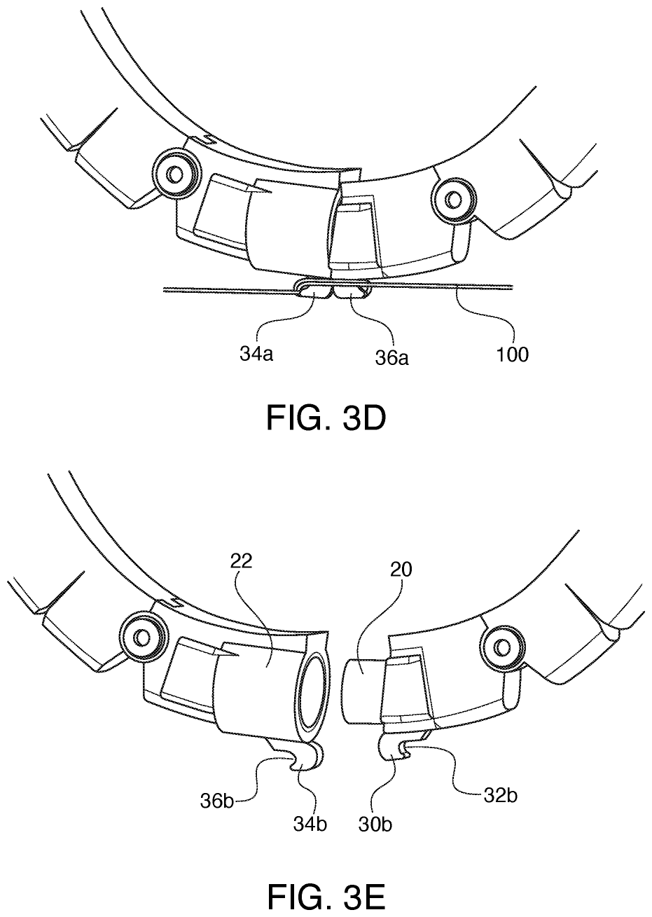

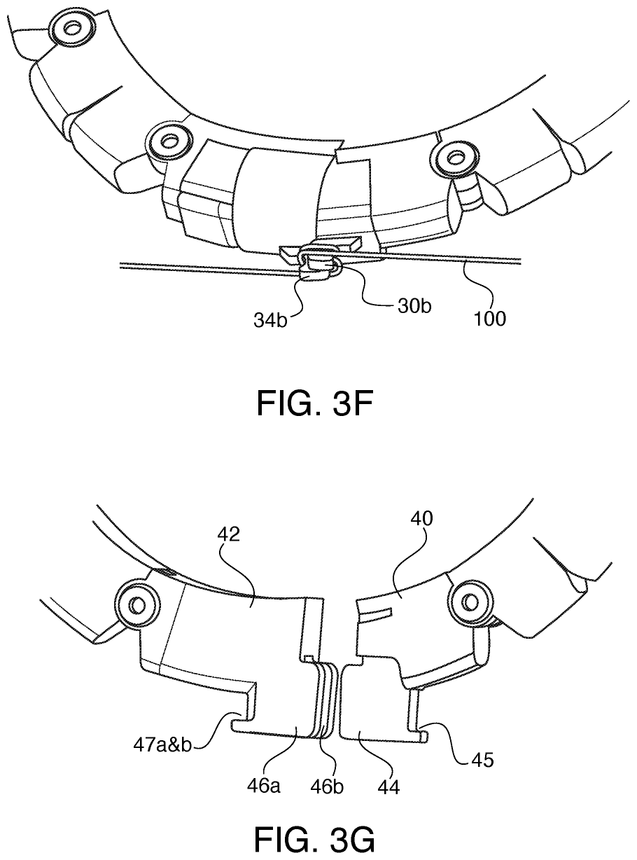

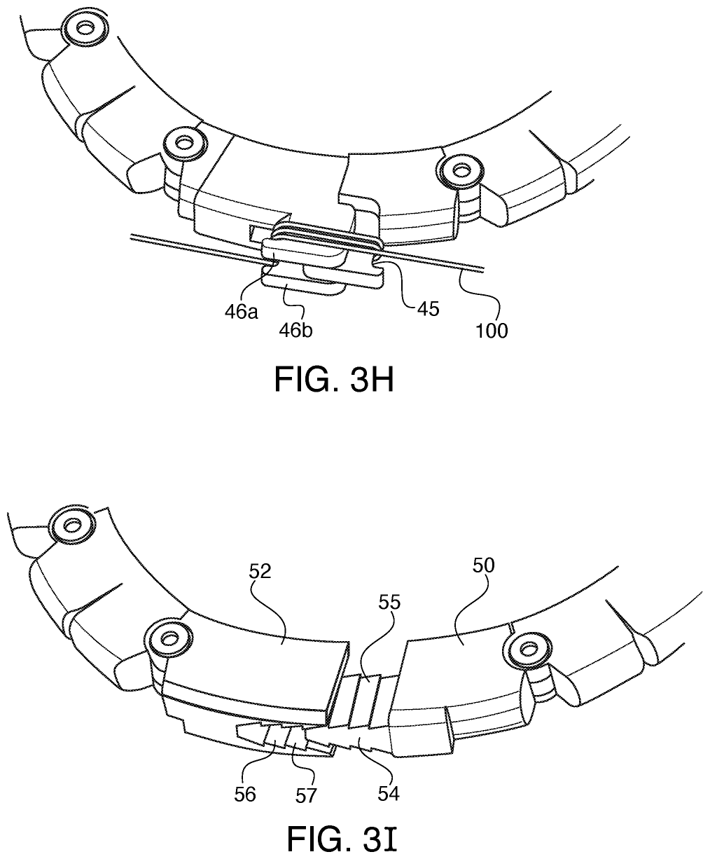

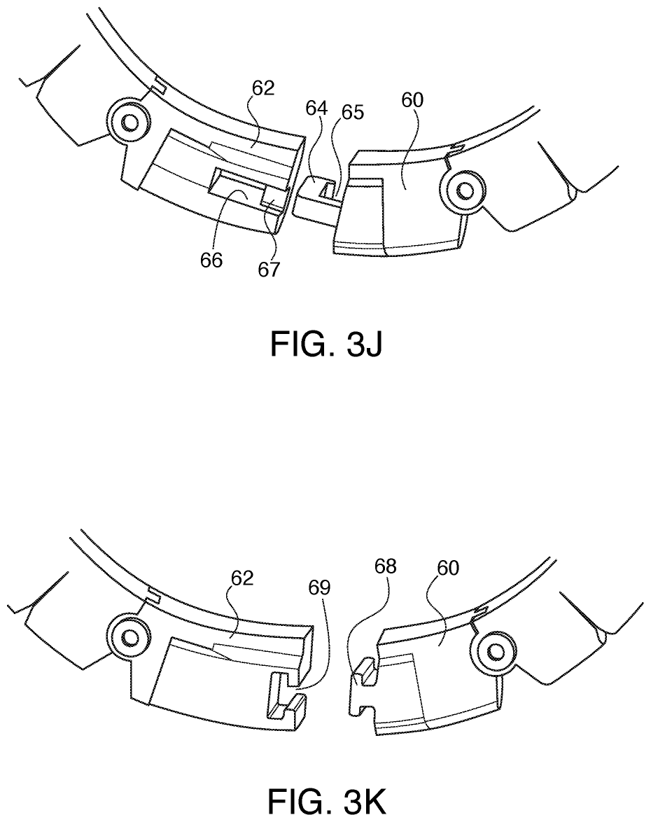

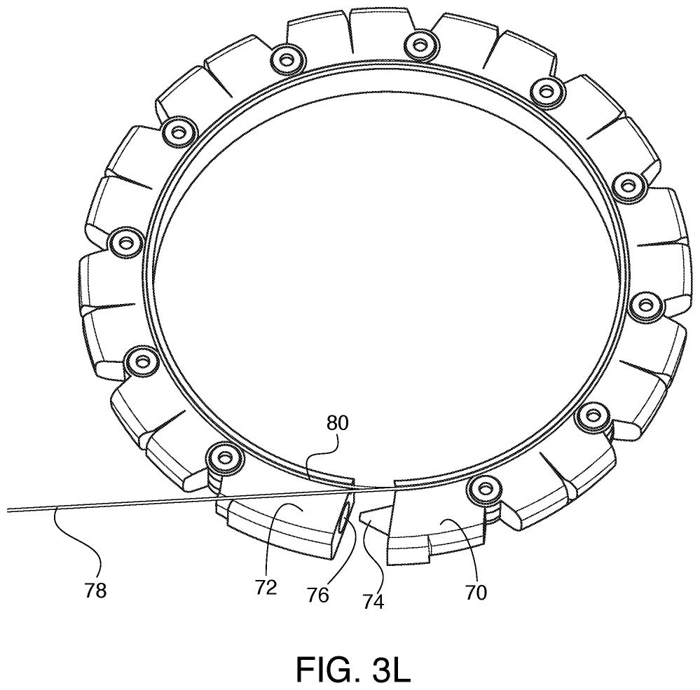

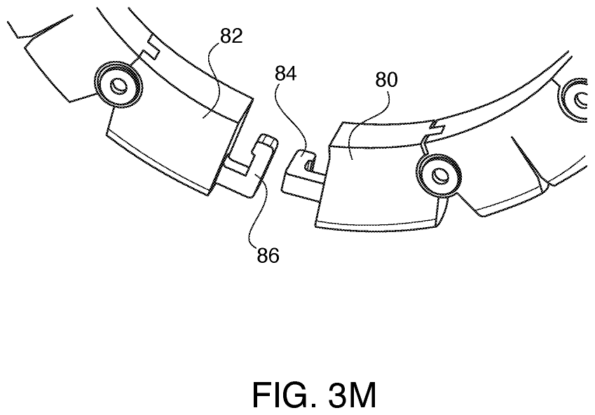

In some embodiments, the ends 20 and 22 are configured to mate with each other and retain the ring 10 in substantially closed and self-supporting configuration. In other embodiments, the ends are brought together through the use of other means, such as a cable or wire. Further details of the ends and the various connection concepts are explained in greater detail below with reference to FIGS. 3A-3M.

As best seen in FIG. 1C, each link 12 of the ring can include suture slots 16A with widened openings 18 that are perpendicular to the circumference of the device and are generally configured for receiving sutures or retraction hooks or the like. As will be appreciated by those of skill in the art, the suture slots 16A are tapered radially inward such that a suture or elastic end of a retraction hook is secured by increased friction within the slot as the suture or elastic end of a retraction hook is radially moved or pulled toward the center of the device or ring.

In another alternative embodiment shown in FIG. 1D, one or more links 26 may not include slots 16A. This link 26 can be used and/or configured or molded for ease in manipulation with a laparoscopic robotic instrument to hold the same in a desired position within the patient during a surgical procedure. The surfaces of the link or links 26 can be textured 28 to allow for easy grasping, retention and/or manipulation. Alternatively, the link can include both the slot 16A and the textured surfaces 28. In fact, in one implementation, the slots 16A could be used by an external tool to assist in the holding and stabilization of the device during use.

In yet a further alternative embodiment shown in FIG. 1E, the ring 10 can have magnets 15 disposed around the circumference of the same. The magnets 15 can be used to manipulate the device position from outside the surgical cavity or can be matched to metallic or magnetic elements in other instruments or devices to allow the device to used to manipulate those instruments. Alternatively, the magnets can be matched to metallic or magnetic elements contained in a gauze so as to affix the gauze in a similar fashion to a “tambourine”. In this manner the ring can be used as a wide-surface-area retractor on intracavity components too delicate to be manipulated or retracted by instruments.

In another embodiment, the magnets 15 can be implemented in the outer surfaces of the hinges or pivot points 14.

In accordance with other embodiments, the outer surfaces of device, or individual links of the same can include small hooks (e.g., Velcro) that could operate to assist in the affixing gauze or the like to the same.

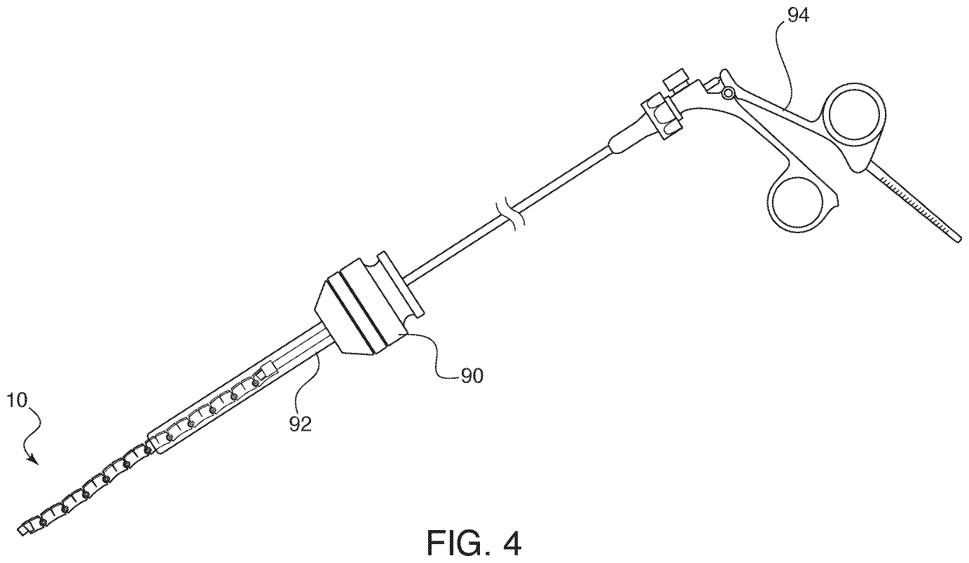

As will be discussed in more detail below, in the exemplary implementation of the present invention, the device 10 is inserted into the patient and then assembled or formed while within the patient (e.g., the abdominal cavity) after passing through, for example, a trocar used for the minimally invasive surgery. (See FIG. 4).

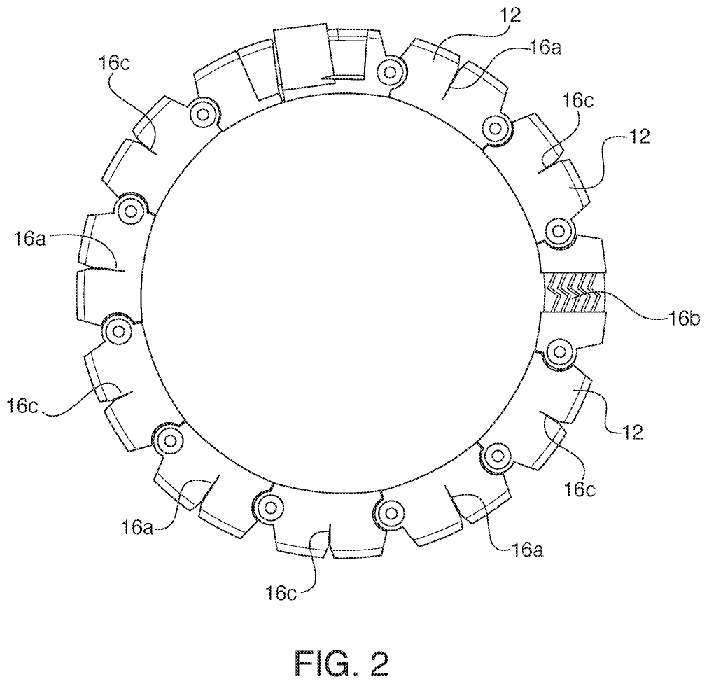

FIG. 2 shows an exemplary embodiment of the retention ring 10 where one of the slots 16A is replaced with springs or wires 16B configured to hold or grasp a suture or the like. It is to be appreciated that one or more slots 16A can be replaced with springs or wires 16B, depending on the choice or preference of application or surgeon. In addition, different size or length slots 16C can be implemented depending on the desired application.

Referring to FIG. 3A, there is a shown one embodiment of the connection mechanism made up of ends 20 and 22. As shown, these ends mate with each other, where end 20 is a post having a magnet 21 on the end thereof and inside the opening or socket 24 is another magnet 23, such that when the ends are brought together, the magnets have sufficient strength/attraction to bring the ends into a mated/connected configuration.

FIG. 3B shows another embodiment of the connection mechanism where the end 20 includes a locking rib 25 that locks end 20 into the socket 24 of end 22. In this embodiment, end 20 is simply moved into the socket 24 and rib 25 “clicks” or fits into snuggly due to friction into the socket 24. Rib 25 can be a flexible o-ring or a semi-rigid ring or the like.

FIGS. 3C and 3D shows another connection mechanism referred to herein as a suture fit closure. In this configuration, post 20 is loosely received into socket 22 such that hooked tabs 30A and 34A are brought adjacent to each other such that hook portions 32A and 36A, respectively, are positioned to allow a suture 100 to be wrapped around the same (FIG. 3D) and thereby holds the ends together and thus forms the ring 10.

FIGS. 3E and 3F show an alternative suture fit arrangement where the hooked tabs 30B and 34B overlap such that the suture 100 is wrapped around the corresponding hook portions 32B and 36B, respectively.

FIGS. 3G and 3H show yet another alternative suture fit arrangement for the connection mechanism where end 40 includes a tab 44 with a hook portion 45, and end 42 includes two tabs 46A and 46B with corresponding hook portions 47A and 47B, respectively. The two tabs 46A and 46B are spaced from each other such that tab 44 fits perfectly between the two tabs. When tab 44 is positioned between tabs 46A and 46B, a suture 100 can wrap around the hook portions (45 and 47A, 47B) of each tab and thereby secure the device shape and form, in this example a ring.

FIG. 31 shows another alternative connection mechanism including an interlocking teeth fit closure. Ends 50 and 52 mate with each other where end 50 includes a tab 54 having angled teeth 55 that fits into the slot 56 on end 52 having similar angled surfaces 57 configured to receive the angled teeth 55 of tab 54. As will be apparent, as tab 54 is inserted into angled slot 56, the angled teeth 55 “click” into each corresponding angled surface 57.

FIG. 3J shows a further alternative connection mechanism including a hook and slot configuration. End 60 includes a hook 64 with a space 65, and end 62 includes a slot 66 having a shoulder 67. When hook 64 is positioned within the slot 66, the shoulder 67 is received within the space 65 thereby securing the hook 64 into the slot 66, and thus securing the device.

FIG. 3K shows yet another alternative connection mechanism for the ends of the ring. In this embodiment, end 60 includes a T-shaped tab 68, and end 62 includes a T-shaped slot 69 configured to receive the T-shaped tab 68 and secure the same in place.

FIG. 3L shows an embodiment for closing the device 10 using a cable or string or suture 78. In this implementation, a cable or suture runs inside a channel 80 that runs the entire length or inner circumference of the ring. The cable/string or suture is fixed at one end in channel 80 (in this example at end 72). Channel 80 is positioned such that when the cable/string or suture is pulled it causes the ring to form the desired shape and ends 70 and 72 to close. An alignment pin 74 is on one end 70 and fits into a hole 76 on the opposing end 76 to maintain the positioning and assure the alignment. In this implementation, once the cable or suture is pulled and the ring formed, the end of the cable or suture 78 can be secured in one of the slots 16 of the device (preferably closer to the end 72 (opposite the end 70 from where the cable/suture comes out of the channel 80. When the surgeon is finished with the procedure, the cable/suture is released or cut from the slot, and the device can be flattened for removal from the surgical site using the same trocar or laparoscopic tool that introduced the same.

In other contemplated implementations, the device can be a flexible material that is in a flaccid state unless and until a wire or cable connected to the same is tensioned, which would result in the device being tensioned and taking the desired shape for use as the intended retraction device.

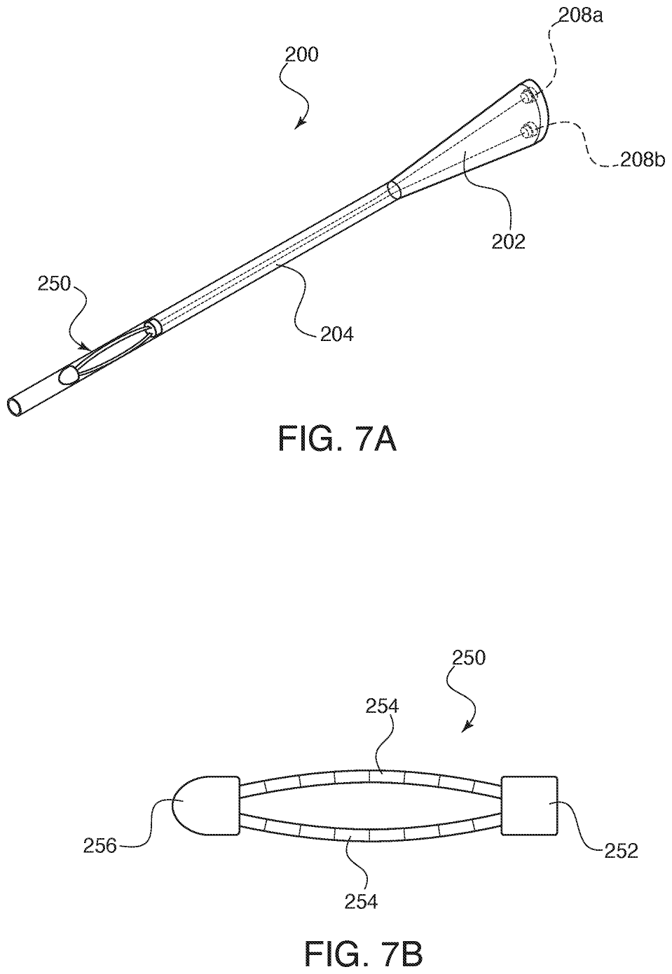

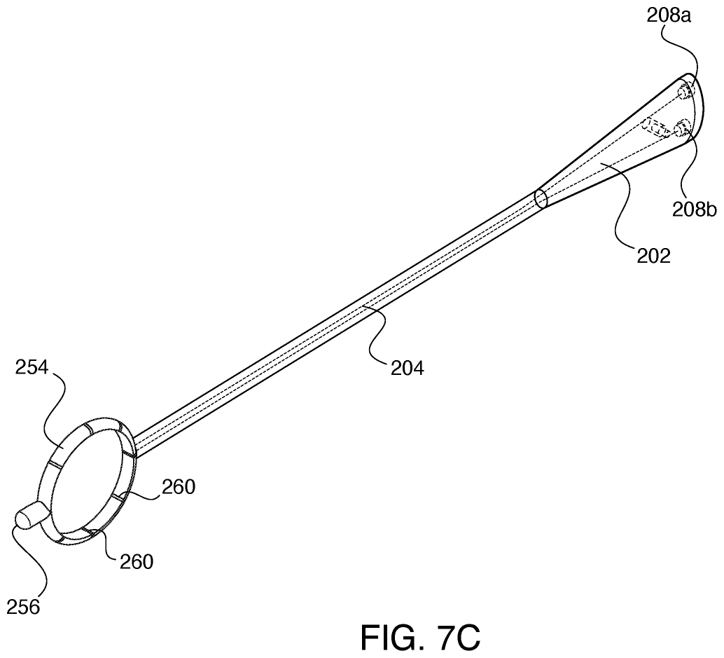

According to yet another implementation of the embodiment of FIG. 3L, if the links 12 are made of a pliable, yet sufficiently rigid material, the number of links can be reduced and made individually larger and can still be substantially flattened for introduction into a surgical site, while the cable/wire system can be used to pull the links together and formed the desired ring once positioned within the patient. (See FIG. 7A-7C)

FIG. 3M shows another connection mechanism utilizing two hooks 84 and 86 extending from ends 80 and 82, respectively. As shown, hook 84 is horizontally positioned while hook 86 is vertically positioned, thus enabling an easy “hooking” of the two to secure the ends of the ring together. It will be appreciated that various shaped hooks in various orientations can be used as an alternative to this exemplary implementation.

FIG. 4 shows one example of a trocar 90 that includes a canula 92 that would be inserted into the patient, and a laparoscopic tool 94 that can introduce the unassembled device 10 through the trocar 90, 92 into the patient at the surgical site.

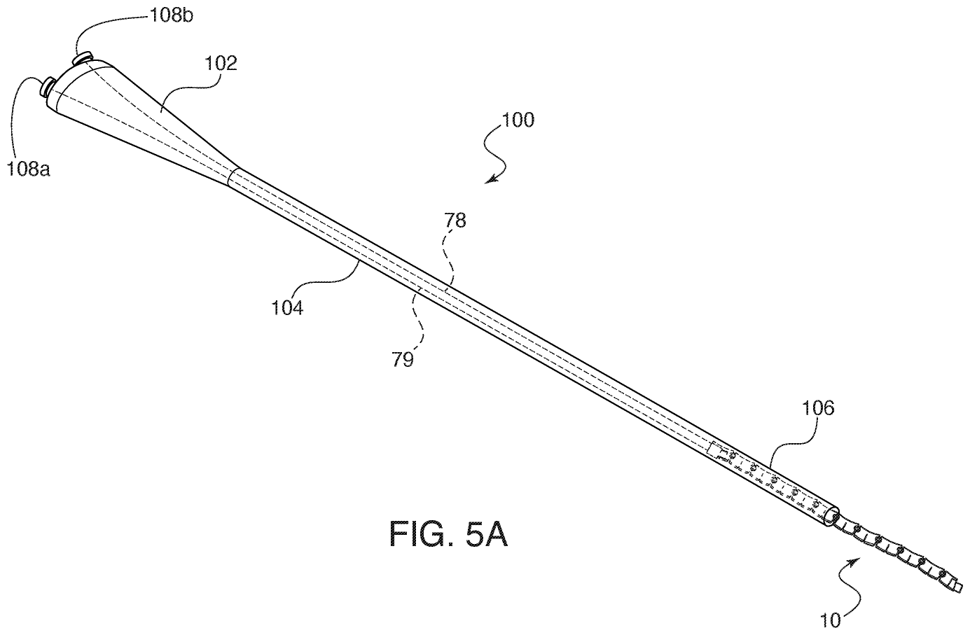

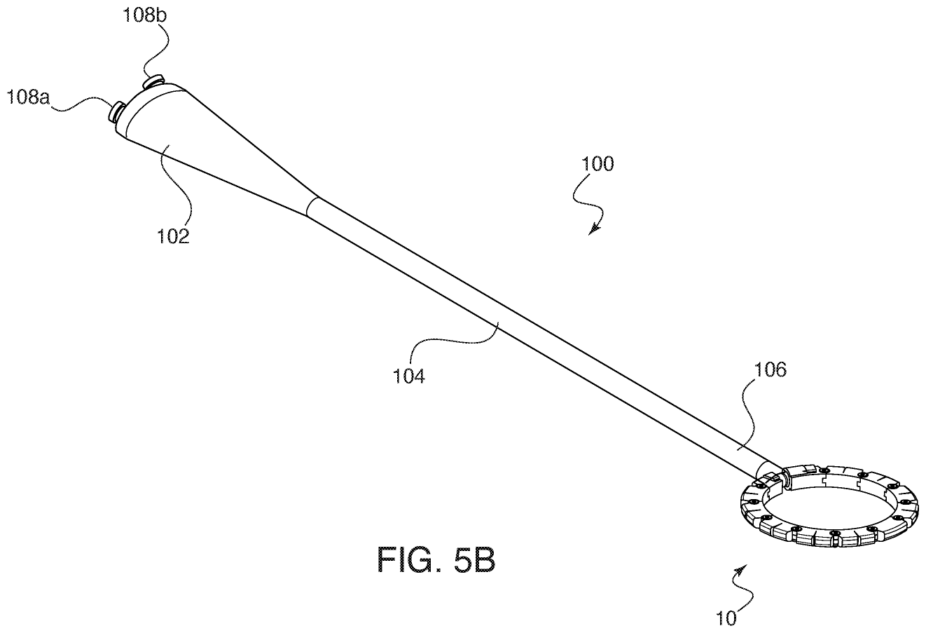

FIGS. 5A-5B show another example of a laparoscopic tool 100 having a shaft 104 with a handle 102 so the unassembled ring 10 can be introduced independently within a surgical site cavity with no portion outside the cavity. In this exemplary embodiment, the ring of FIG. 3L can be used with the wire/cable disposed within the same, where the wire/cable is connected to one of the dials 108A or 108B. When the dial is rotated, it can pull on or increase the tension of the cable 78 and thus form the ring at the end 106 of the tool 100. The device 10 is now ready to be used within the patient. In other contemplated embodiments more than one wire/cable can be used such that each dial 108A and 108B controls one wire/cable. For Example, a second wire/cable 79 can be positioned in a channel that is disposed radially outward from the channel 80 along the outer circumference of the ring (FIG. 3L) and this wire/cable can be used to control the opening and/or closing of the slots 16A, or springs 16B that hold the sutures or retention hooks, or the like.

In accordance with a further embodiment, the device 10 at the end of tool 100 can be formed of a material that can be collapsed on itself (i.e., flattened) without the use of cables or sutures, and inserted through a trocar. The material would be elastic and sufficiently rigid with a shape memory such that once the collapsed ring passed through the end of the trocar, the ring would snap back into its desired/preformed shape. FIGS. 7A-7C show this concept, where the device 250 is made of a flexible material 254 such that it can be collapsed on itself and inserted into the trocar 204 of the tool 200. Using the handle 202, and at least on control 208a or 208b, the ring 250 can travel along the trocar 204. In one implementation, the flexible material of the device 254 is positioned between a rigid distal end 256 and a proximal end 252 that is engaged by control lines (not shown) that lead to the handle 202 and controls 208. In this configuration, when the proximal end 252 clears the end of the trocar, the device 254 would “snap” or “pop” into the ring shape due to the shape memory of the material. FIG. 7C shows the device 254 at the end of the trocar, having one or more slits 260.

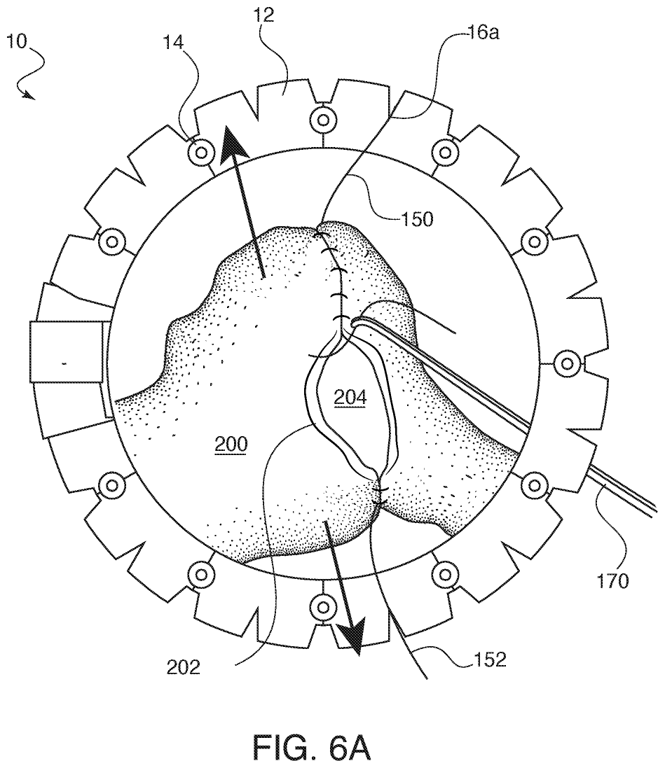

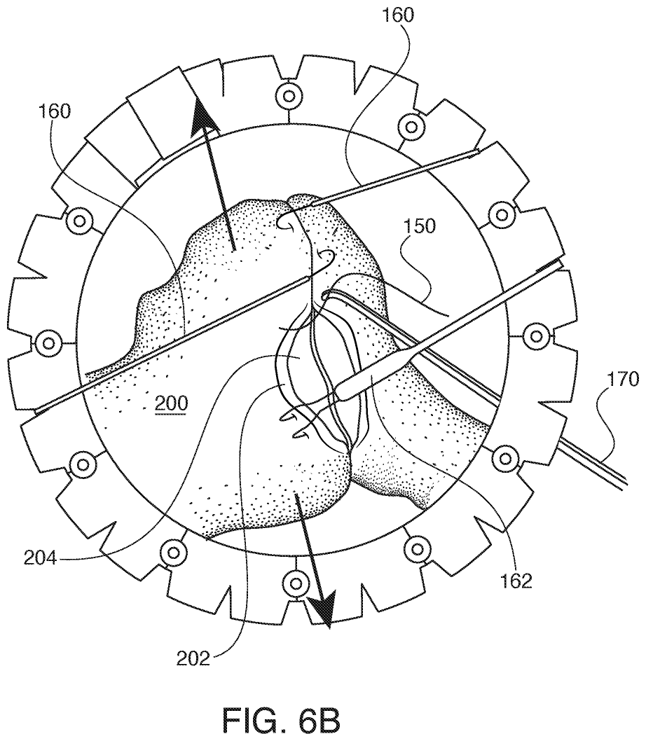

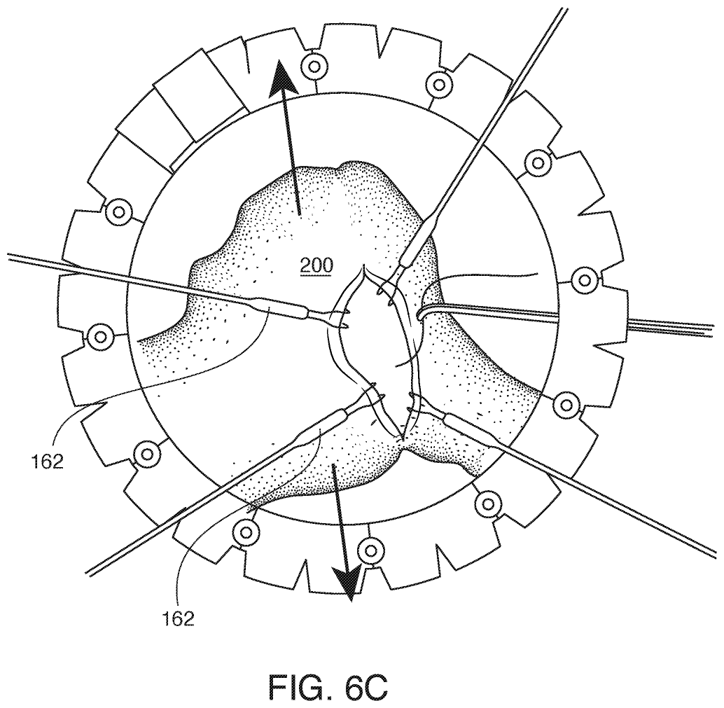

FIGS. 6A-6C show non-limiting examples of how the retention device 10 can be used in different surgical applications. Referring to FIG. 6A, a typical suturing application/method is shown. Initially, a first suture 150 is places through the tissue 200 at one end of the defect 202 to be closed. The first suture 150 is then trapped or secured in a slot 16A in the ring 10. As mentioned above, the slot 16A is generally configured such that when the suture 150 is pulled over the device and radially inward into the slot, the friction alone holds the suture in place. In the alternative embodiment mentioned with reference to FIGS. 5A-5B, if a second wire/cable is used to open and close the slots 16A, 16C or springs 16B, then the rotation of the corresponding knob 108 would close the slot on the suture. Once the first suture 150 is secured in the ring, a second suture 152 is placed through the tissue at the opposite end of the defect. The second suture is then also secured into a slot 16A. The defect in the tissue can now be stretched along its axis and the surgeon can now suture the defect using the appropriate tool or tools 170.

FIG. 6B shows an example of the use of retraction hooks 160 and 162 in conjunction with the device to secure and retain the ends of the tissue defect 202 in place while the surgeon sutures 150 the same using the appropriate tool or tools 170.

FIG. 6C shows another example of the use of retraction hooks 162 in conjunction with the device, where the hooks hold an opening 204 in the tissue open while the surgeon repairs, for example, a vessel exposed by the opening 204 in the tissue 200.

In addition to the above examples, there are many other applications for the retraction device 10 of the present invention and its ability to be formed inside the patient. By way of further example, the ring can be designed to accommodate tissue into the slots 16A so tissue can be directly attached to the device frame and thus manipulated. Alternatively, the device 10 can come in a kit which includes a multitude of hook elements (e.g., 160, 162) each connected to a rubber string which can be trapped in the ring's slits and act as temporary positioning anchors.

Finally, additional elements or the device material itself can act to capture/hold sutures which will allow for placement of a number of sutures at one time into the surgical cavity. For example, at the commencement of a procedure the scrub technologist could connect all of the sutures to be used for a given procedure into the slots 16A, 16C of the device 10 prior to the ring being inserted into the patient. Then the device 10, with already connected sutures can be introduced into the surgical cavity thereby making them available to the surgeon when needed without the surgeon having to wait for each suture to be handed or introduced separately. Similarly, at the conclusion of the procedure, all of the used sutures can be attached to the device to allow for easy, single action, extraction of a number of sutures. This can lead to improved safety since each suture doesn't have to be removed separately and potentially lost. These elements for suture capture can be separate features and composed of a soft material or rubber attached to the device or be the ring material itself. For example, silicone could be used for this application.

While there have been shown, described and pointed out fundamental novel features of the present principles, it will be understood that various omissions, substitutions and changes in the form and details of the methods described and devices illustrated, and in their operation, may be made by those skilled in the art without departing from the spirit of the same. For example, it is expressly intended that all combinations of those elements and/or method steps which perform substantially the same function in substantially the same way to achieve the same results are within the scope of the present principles. Moreover, it should be recognized that structures and/or elements and/or method steps shown and/or described in connection with any disclosed form or implementation of the present principles may be incorporated in any other disclosed, described or suggested form or implementation as a general matter of design choice. It is the intention, therefore, to be limited only as indicated by the scope of the claims appended hereto.

It should also be understood that the example embodiments disclosed and taught herein are susceptible to numerous and various modifications and alternative forms. Thus, the use of a singular term, such as, but not limited to, “a” and the like, is not intended as limiting of the number of items. Furthermore, the naming conventions for the various components, functions, parameters, thresholds, and other elements used herein are provided as examples, and can be given a different name or label. The use of the term “or” is not limited to exclusive “or” but can also mean “and/or”.

What is claimed is:

1. A retraction device for minimally invasive surgery comprising:
a plurality of links pivotally connected to each other forming a chain of links having opposing ends; and
a connection mechanism positioned at each of the opposing ends and configured to connect the opposing ends and cause the plurality of links to form the retraction device;
wherein the plurality of links are introduced into a patient in a substantially flat configuration prior to engagement of the connection mechanism.

2. The retraction device of claim 1, wherein at least one of the plurality of links further comprises a slit for receiving and securing at least one of a suture or a retraction hook.

3. The retraction device of claim 2, wherein the slit has a variable size and at least two links of the plurality of links have slits of a different size with respect to each other.

4. The retraction device of claim 1, wherein at least one of the plurality of links comprises a textured surface to assist in handling and manipulation of the retraction device during use.

5. The retraction device of claim 1, wherein the connection mechanism comprises magnets disposed in or on the opposing ends of the plurality of links.

6. The retraction device of claim 1, wherein the connection mechanism comprises a hook disposed on each of the opposing ends of the plurality of links, wherein a suture is wrapped around each hooks on each opposing end to hold and secure the opposing ends together.

7. The retraction device of claim 1, wherein the connection mechanism comprises a socket at one of the opposing ends and a press fit end having a locking rib such that when the locking rib engages the socket, the opposing ends are secured together.

8. The retraction device of claim 1, further comprising:
a channel formed in the plurality of links; and
a wire positioned in the channel and being fixed at one of the opposing ends and extending out of the channel at the other of the opposing ends, the wire enabling the formation of the retraction device by pulling on the extended end which brings the opposing ends together.

9. A retraction device for use inside a patient during minimally invasive surgery, the retraction device comprising:
at least two links connected to each other and having opposing ends, each of the at least two links having at least one slit configured to receive and secure a suture or a retraction hook, wherein the at least two links can be laparoscopically introduced into a patient in a substantially flat configuration.

10. The retraction device of claim 9, further comprising a connection mechanism positioned at each of the opposing ends and configured to connect the opposing ends and cause the plurality of links to form the retraction device.

11. The retraction device of claim 9, further comprising:
a channel formed in the at least two links; and
a wire positioned in the channel and being fixed at one of the opposing ends and extending out of the channel at the other of the opposing ends, the wire enabling the formation of the retraction device by pulling on the extended end which brings the opposing ends together.

12. The retraction device of claim 9, wherein at least one of the at least two links includes a textured surface to assist in handling and manipulation of the retraction device during use.

13. The retraction device of claim 10, wherein the connection mechanism comprises magnets disposed in or on the opposing ends.

14. The retraction device of claim 10, wherein the connection mechanism comprises a hook disposed on each of the opposing ends, wherein a suture is wrapped around each hook on each opposing end while inside the patient to hold and secure the opposing ends together.

15. The retraction device of claim 10, wherein the connection mechanism comprises a socket at one of the opposing ends and a press fit end having a locking rib disposed around the same such that when the locking rib engages the socket, the ends are secured together.
